(19) United States
(12) Patent Application Publication
(10) Pub. No.: US 2026/0024377 A1
(43) Pub. Date: Jan. 22, 2026

(54) METHOD, APPARATUS, AND COMPUTER PROGRAM PRODUCT FOR FACE LIVENESS DETECTION

(71) Applicant: Candour Oy, Oulu (FI)

(72) Inventors: Zinelabidine BOULKENAFET, Oulu (FI); Olli SILVÉN, Oulu (FI)

(21) Appl. No.: 18/992,516

(22) PCT Filed: Jul. 6, 2023
(86) PCT No.: PCT/FI2023/050430

§ 371 Date: Jan. 8, 2025

(30) Foreign Application Priority Data
Jul. 8, 2022 (FI) 20225646

(51) Int. Cl. G06V 40/40; G06V 10/56; G06V 10/74; G06V 10/75; G06V 40/16

(52) U.S. Cl. CPC G06V 40/45; G06V 10/56; G06V 10/759; G06V 10/761; G06V 40/168

(57) ABSTRACT

A method, apparatus, and computer program product for face liveness detection are disclosed. The method comprises: obtaining one or more color image data frames, each color image data frame depicting a face of a subject; identifying a plurality of skin regions; extracting a skin region data set from each one of the plurality of identified skin regions; computing a plurality of color distributions, each color distribution being computed on the basis of one of the plurality of skin region data sets; determining at least one distance between the plurality of color distributions; if the at least one distance is greater than a liveness threshold, detecting positive liveness of the subject, and else detecting negative liveness of the subject; and outputting the detected positive or negative liveness.

### TECHNICAL FIELD

The present solution generally relates to a method, an apparatus, and a computer program product for face liveness detection.

### BACKGROUND

Biometric face identification and verification are subject to various kinds of presentation attacks. Static two-dimensional attacks employ photographs or pictures presented on a display. Dynamic two-dimensional attack schemes employ sequences of video replayed on a display or injected as an input from a virtual camera. Static three-dimensional attacks utilize 3D printer reproductions of faces, and dynamic three-dimensional attacks can be implemented using latex masks or make-up, for example.

Some biometric face verification systems attempt to combat presentation attacks with increasingly sophisticated and expensive anti-spoofing technologies. At the same time, it is desirable that the biometric verification, including the anti-spoofing, exhibits a low false negative rate and performs rapidly, both to avoid inconvenience to the user. Many anti-spoofing technologies do not fulfil both of the requirements for accurate performance and quick operation.

Many smartphones are equipped with 3D infrared scanners that enable discriminating between a flat image and a three-dimensional face. To determine that the face belongs to a living subject, liveness detection techniques may be employed. Some two-dimensional image biometric verification systems employ a challenge-response liveness detection method that asks the user to collaborate, e.g., by turning one's head. Alternatively, the liveness of the subject may be determined from eyeblinks, or by extracting a heart rate signal or an electrocardiogram signal from the face of the subject. However, the above liveness detection methods require several seconds of measuring, and a quicker solution would be desirable.

### SUMMARY OF THE INVENTION

The scope of protection sought for various embodiments of the invention is set out by the independent claims. Various embodiments are disclosed in the dependent claims. Although various aspects of the embodiments are set out in the independent claims, other aspects comprise other combinations of features from the described embodiments and/or the dependent claims with the features of the independent claims, and not solely the combinations explicitly set out in the claims.

### BRIEF DESCRIPTION OF THE DRAWINGS

FIG. 1 illustrates an example scenario and embodiments of a system for face liveness detection;

FIG. 2 is a schematic diagram depicting embodiments of an apparatus;

FIG. 3 is a flow chart illustrating embodiments of a method for face liveness detection; and

FIG. 4 illustrates embodiments related to frames, skin regions, and color distributions.

### DETAILED DESCRIPTION OF THE INVENTION

The following description and drawings are illustrative and are not to be construed as unnecessarily limiting. The specific details are provided for a thorough understanding of the disclosure. However, in certain instances, well-known or conventional details are not described in order to avoid obscuring the description. In this specification, reference to “one embodiment” or “an embodiment” means that a particular feature, structure, or characteristic described in connection with the embodiment is included in at least one embodiment of the disclosure. References to an embodiment can be, but are not necessarily, references to the same embodiment in the present disclosure.

The present disclosure relates to a method, an apparatus, and a computer program product for liveness detection. Liveness detection or anti-spoofing serves to detect whether a subject identifying or authenticating with a biometric identifier is a genuine, living being or a fake representation. In the latter case, a presentation attack, also known as a spoofing attack, may be detected. The subject is usually a human subject.

The disclosed solution is based on detecting color differences on the face of a subject. The color differences originate from pulse and respiration dependent oxygenation changes of blood that circulates in the capillaries close to the skin. The changes may be easiest to detect in locations where there is good blood circulation near the surface of the skin, and the face is such a location.

The physics of the color differences is based on the following general principles. A camera detects reflected light that depends not only on the ‘real’ color of the skin but also on the wavelength content of illumination. Concerning blood, hemoglobin of red blood cells absorbs blue and green light and reflects red light when bound to oxygen. Consequently, oxygenated blood appears red. A higher level of oxygenation makes it appear an even brighter red.

Veins may sometimes appear blue through the skin, and the explanation to the blueish hue is that blue light penetrates tissue much less than red light. When the veins are located deeper in the tissue, the balance between blue and red is altered as less red light is reflected back as it is partly absorbed by tissue on its way back and forth. While part of it is reflected, it is reflected from tissue, and the component reflected from blood is highly attenuated.

Changes of color caused by blood pulses take place at slightly different times in different areas of the face. The change is first apparent under the eyes, then on the cheeks, and finally on the forehead. It has been discovered that color differences between different areas of the face and/or changes in the color of the same area over time are indicative of the presence of a pulse. For example, there may be color changes in the patches of skin under the eyes and at the cheeks between images of a living person. Alternatively or additionally, there may be color differences between a patch of skin under the eye and a patch of skin on the cheek in the same image.

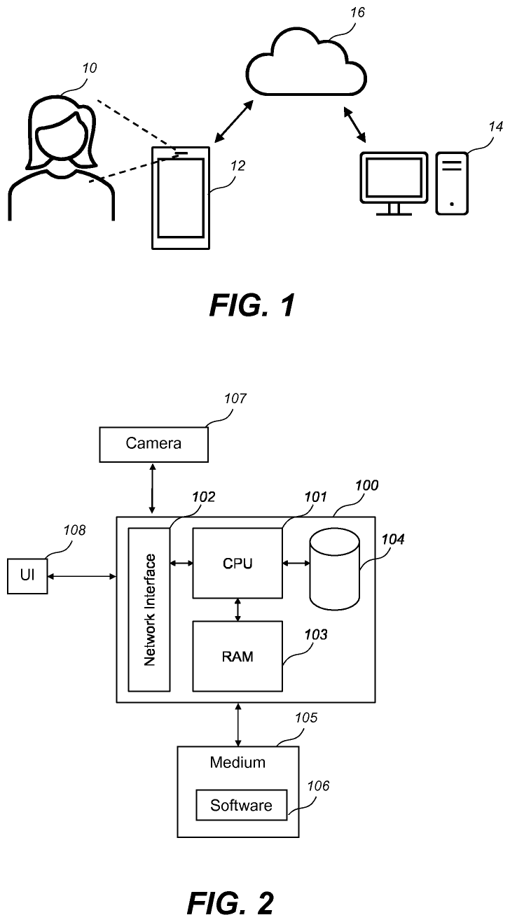

FIG. 1 illustrates an example scenario and system for face liveness detection. The system may comprise a user device 12 and a server 14. The user device 12 is a computing device, and the server is another computing device that is connectable to the user device via a network 16. The user device 12 may be a personal computer, a mobile device, such as a smartphone, tablet computer, laptop, smart watch, or another mobile computing device. A user 10 may wish to biometrically identify themselves to perform an action using the user device 12 and/or the server 14, and/or to gain access to an application or data stored in the user device 12 and/or the server 14. Biometric identification may be passed using a biometric identifier, also known as a biometric sample, such as the face of the user. As an example, the user may wish to sign a document or attend an online exam using their face as the biometric identifier to prove their identity. The user may use a camera of the user device 12 to take a photo or video of their face, and the photo/video may be analyzed to identify the user. To prevent unauthorized parties from identifying as the user 10, liveness detection or anti-spoofing may be performed to distinguish the living user 10 from a presentation attack.

For example, when the user 10 wishes to access an application on the user device 12, the biometric identification and the liveness detection may be performed by the user device 12 alone on the basis of the photo/video captured by the user 10 using the user device 12. If the identification and liveness detection succeed, the user device allows the user 10 to access the application with the user device 12.

In another example, the user wishes to biometrically identify themselves to gain access to a building. The user device 12 executing an access control application may send the results of the identification and liveness detection to the server 14 executing an access control program, and the server 14 executing the access control program may grant the user 10 access to the building e.g. by sending a command to unlock an electric lock of a door of the building.

In another example, the user wishes to attend an online exam that uses biometric invigilation. The user device 12, being e.g. a personal computer or laptop of the user, may send video captured by an integrated or external camera to the server 14. The server 14 may perform the identification and liveness detection, and grant the user access to an exam platform executing on the server 14.

In another example, the user wishes to sign a document using their face as a biometric identifier. The user device may send photo/video data captured by the user device 12 to the server 14. The server 14 may perform the identification and liveness detection, and send the results of the identification and liveness detection to the user device 12. The user device 12 may receive the results and allow the user to sign a document using the user device 12.

FIG. 2 is a schematic diagram depicting embodiments of an apparatus. The apparatus 100 may be a general-purpose computer, such as the server 14 of FIG. 1. Alternatively, the apparatus may be the user device 12 of FIG. 1. The apparatus 100 may include at least one processor 101, such as a central processing unit (CPU) and/or a graphics processing unit (GPU). The apparatus 100 may include at least one memory 103, 104, such as random access memory (RAM) 103, and/or non-volatile memory 104. The apparatus may be but need not be dedicated hardware. The apparatus may be a virtual machine. The method, described in more detail below, may be executed as a containerized application using operating system (OS)-level virtualization.

The apparatus 100 may comprise a network interface 102 for communicating with other devices via a network. The apparatus 100 may be located in a data center and accessible via the network through the network interface 102. The network interface may comprise one or more network interfaces, such as a cellular network interface, an Internet of Things (IoT) network interface, a personal area network (PAN) interface, and other suitable network interfaces.

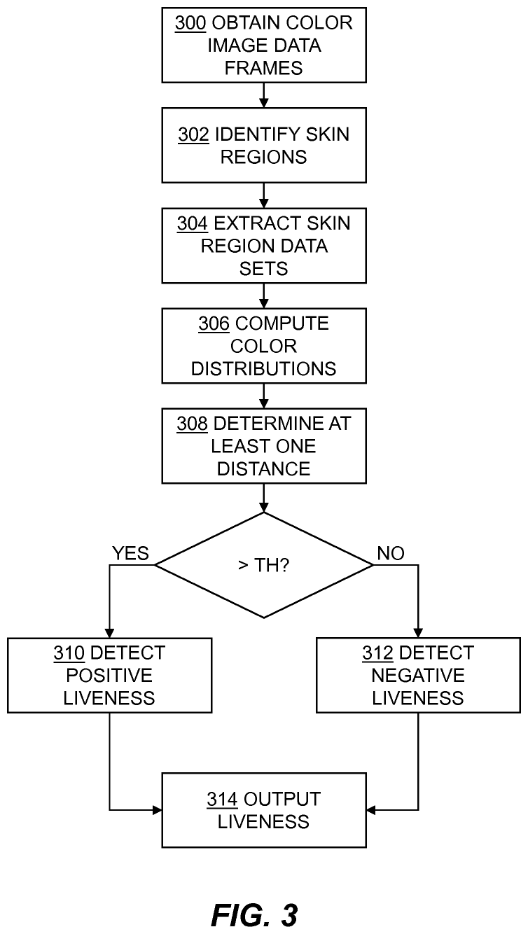

FIG. 3 is a flow chart depicting embodiments of a (computer-implemented) method for face liveness detection. The method of FIG. 3 may be performed by the user device 12 of FIG. 1. Alternatively, the method of FIG. 3 may be performed by the server 14 of FIG. 1. The method comprises obtaining 300 one or more color image data frames, each color image data frame depicting a face of a subject 10; identifying 302 a plurality of skin regions by one of:
a) identifying at least one skin region in each of a plurality of color image data frames;
b) identifying a plurality of skin regions in a single color image data frame;
c) identifying a plurality of skin regions in each of a plurality of color image data frames;
extracting 304 a skin region data set from each one of the plurality of identified skin regions;
computing 306 a plurality of color distributions, each color distribution being computed on the basis of one of the plurality of skin region data sets; determining 308 at least one distance between the plurality of color distributions; if the at least one distance is greater than a liveness threshold, detecting 310 positive liveness of the subject, and else detecting 312 negative liveness of the subject; and outputting 314 the detected positive or negative liveness.

Technical effects of the invention include increased speed of liveness detection and detection of presentation attacks. When known solutions require several seconds of video data depicting heart rate signals and/or movements of the subject's head, the present disclosure allows for liveness detection in a shorter time frame. The sensitivity and specificity of the detection may also be improved, especially when considered with respect to the time taken for the detection. Further, the method may be computationally more efficient, as a reduced number of frames may be processed when compared to the processing of several seconds of video data. Detecting the color differences and thus the liveness of the subject based on color distributions is also very noise resistant. With increased sensitivity of mobile device camera sensors, the color differences may be detected using color image data frames captured by a smartphone camera.

The apparatus 100 of FIG. 2 may be configured to perform the method of FIG. 3 or any of its embodiments. The apparatus 100 may comprise means for performing the method of FIG. 3 or any of its embodiments. According to an aspect, an apparatus 100 comprises at least one processor 101, at least one memory 103, 104 including computer program code, the at least one memory 103, 104 and the computer program code configured to, with the at least one processor 101, cause the apparatus 100 to perform the method of FIG. 3 or any of its embodiments. As mentioned above, the apparatus 100 may be the user device 12 or the server 14 of FIG. 1.

Referring again to FIG. 2, a computer program product or a computer-readable medium 105 comprises computer program code 106 configured to, when executed by at least one processor 101, cause an apparatus 100 or a system to perform the method of FIG. 3 or any of its embodiments. In an embodiment, the computer-readable medium is a non-transitory computer-readable medium.

The method of FIG. 3 comprises obtaining 300 one or more color image data frames. Each color image data frame depicts a face of a subject. In an embodiment, the obtaining comprises measuring the color image data frames e.g., by the camera 107 illustrated in FIG. 2. In an embodiment, the apparatus 100 or the system comprises the camera 107 configured to measure the one or more color image data frames. The camera may comprise a visible spectrum camera, an infrared scanner, a near-infrared camera, and/or a thermal camera. The camera may be configured to measure, and/or the color image data frames may comprise one or more of: visible spectrum image data, ultraviolet image data, infrared image data, near-infrared image data, and thermal image data. The meaning of the term ‘color’ is herein understood to cover electromagnetic spectra of the light received from the face of the subject also beyond the human visible spectrum. The use of image data beyond the visible electromagnetic spectrum may improve detection of color differences caused by a pulse from the face of the subject. Further, especially the near-infrared image data may be advantageous for detecting color differences in dark-skinned individuals. For example, the one or more color image data frames may comprise visible spectrum image data in red, green, and blue (RGB) channels, and infrared image data in an infrared channel. As another example, the blue channel of RBG image data may be replaced with the infrared channel such that the one or more color image data frames may comprise visible spectrum image data in the red and green channels, and infrared image data in the infrared channel. The blue channel may only contain very little relevant information with respect to color changes caused by the pulse and may thus be removed to improve computational efficiency.

Alternatively, or additionally, the obtaining may comprise reading the one or more color image data frames from the at least one memory of the apparatus. When the apparatus is the server 14 of FIG. 1, the obtaining may comprise receiving the one or more color image data frames from the user device 12. The user device 12 may acquire the one or more color image data frames e.g., using its camera, and transmit the color image data frames to the server 14 e.g., via the network 16 and/or by a network interface of the user device. The server 14 may receive the one or more color image data frames via the network 16 and/or by a network interface of the server 14.

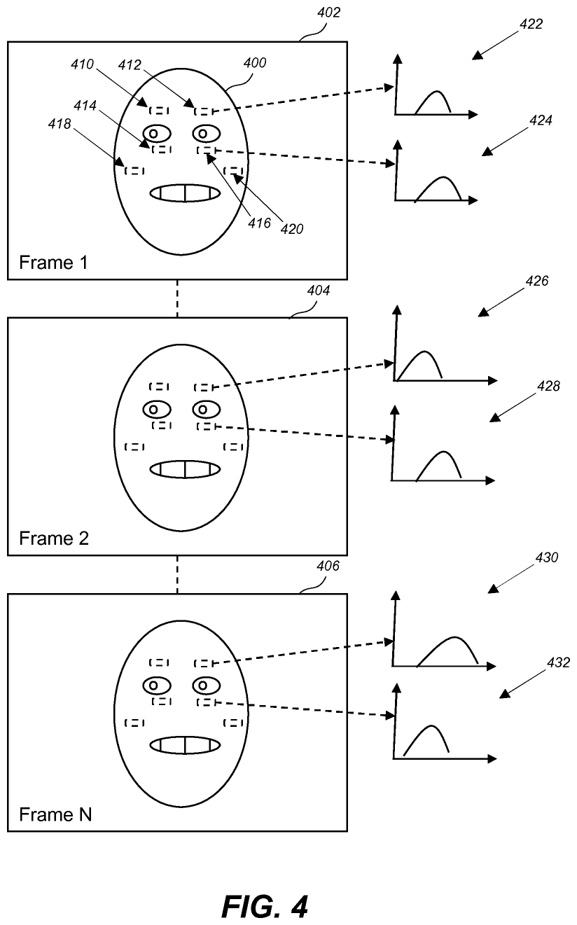

The method of FIG. 3 further comprises identifying 302 a plurality of skin regions. FIG. 4 illustrates some examples of the plurality of skin regions of subject 400 depicted in frames 402, 404, and 406. The plurality of skin regions may comprise one or more forehead regions, such as a left forehead region 410 and a right forehead region 412, one or more under-eye regions, such as a left under-eye region 414 and a right under-eye region 416, and/or one or more cheek regions, such as a left cheek region 418 and a right cheek region 420, as illustrated in FIG. 4. The terms ‘left’ and ‘right’ only serve the purpose of distinguishing the two respective regions from one another; it is not relevant whether they refer to the true left or right of the subject, or to an observer's left and right when viewing a color image data frame. The latter approach (observer's left and right) is adopted herein when discussing the skin regions illustrated in FIG. 4.

The plurality of skin regions may be predetermined, and the predetermined skin regions may be stored in the at least one memory, for example. The identifying may comprise tracking the face of the subject and/or identifying locations of one or more anatomical features or landmarks on the face of the subject. The landmarks may represent the eyebrows, eyes, nose, lips, and/or jawline of the subject. Face tracking and identification of landmarks are generally known in the art and disclosed e.g., in “Real-time face alignment: evaluation methods, training strategies and implementation optimization”, a Master's thesis by Constantino Alvarez Casado, published on 2020 Dec. 18. The plurality of skin regions may be identified on the basis of the identified locations of the landmarks. For example, the skin regions may be bounded by specific landmarks, and/or defined by predetermined distances from the landmarks. As an example, a forehead skin region may be bounded by hairline landmarks and eyebrow landmarks. As another example, an under-eye region may cover a predetermined distance downwards from eye landmarks.

Identifying the plurality of skin regions may be performed by identifying at least one skin region in each of a plurality of color image data frames. The at least one skin region may comprise (only) one skin region. Here, the at least one skin region refers to the same at least one skin region. For example, the (same) at least one skin region may be the left forehead skin region 410 that is identified in each of frames 402, 404, and 406. As another example, the at least one skin region may comprise a plurality of skin regions, such as forehead skin regions 410 and 412. The (same) plurality of skin regions may be identified in each of frames 402, 404, and 406, for example. A benefit of identifying the at least one skin region in each of a plurality of color image data frames is that information on color changes that occur over time in the at least one skin region is obtained.

A plurality of skin regions may together form a composite skin region. For example, the left and right forehead skin regions 410, 412 may together form a forehead skin region 410, 412. The forehead skin region may then be considered as one skin region. Composite skin regions may provide benefits in relation to how the skin regions are identified. For example, it may be computationally more accurate and/or efficient to identify two or more parts of a skin region separately, e.g., based on the landmarks of the subject's face, and then join them together.

Alternatively, identifying the plurality of skin regions may be performed by identifying a plurality of skin regions in a single color image data frame. Here, the plurality of skin regions refers to different skin regions. Each skin region of the plurality of different skin regions may be identified in a single color image data frame. For example, both of the under-eye skin regions 414, 416 and the composite forehead skin region 410, 412 may each be identified in the frame 402. A benefit of identifying a plurality of skin regions in a single color image data frame is that information on color differences between different areas of the face at one time instant is obtained.

Alternatively, identifying the plurality of skin regions may be performed by identifying a plurality of skin regions in each of a plurality of color image data frames. The same skin regions may thus be identified in a plurality of frames. For example, both of the under-eye skin regions 414, 416 and the composite forehead skin region 410, 412 may each be identified in each of the frames 402, 404 and 406. Information on both the color changes that occur over time in the plurality of skin regions, and color differences between different areas of the face at one time instant, is obtained.

The method of FIG. 3 further comprises extracting 304 a skin region data set from each one of the plurality of identified skin regions. The skin region data sets are extracted from the skin regions in the one or more color image data frames. One skin region data set is extracted for each skin region identified in the one or more color image data frames.

For example, when at least one skin region is identified in each of a plurality of color image data frames 402, 404, 406, a first forehead skin region data set may be extracted from the (composite) forehead skin region 410, 412 of frame 402, a second forehead skin region data set may be extracted from the forehead skin region of frame 404, and a third forehead skin region data set may be extracted from the forehead skin region of frame 406. As another example, a first right cheek skin region data set may be extracted from the right cheek skin region 416 of frame 402, a second right cheek skin region data set may be extracted from the right cheek skin region of frame 404, and a third right cheek skin region data set may be extracted from the right cheek skin region of frame 406.

In an example wherein a plurality of skin regions is identified in a single color image data frame 402, a forehead skin region data set may be extracted from the (composite) forehead skin region 410, 412 of frame 402, a left cheek skin region data set may be extracted from the left cheek skin region 414 of the same frame 402, and a right cheek skin region data set may be extracted from the right cheek skin region 416 of the same frame 402.

Continuing the previous example wherein now a plurality of skin regions are identified in a plurality of color image data frames 402, 404, a further forehead skin region data set may be extracted from the forehead skin region of frame 404, a left cheek skin region data set may be extracted from the left cheek skin region of frame 404, and a right cheek skin region data set may be extracted from the right cheek skin region of frame 404.

Each skin region data set may contain image data of the color image data frame that depicts the skin region in the color image data frame. The one or more color image data frames may be in any suitable color image format, commonly in a raster image format or video frame format. Color images and video are often encoded using the RGB color space with one channel for each of the red, green and blue components, however, any suitable color space such as hue, saturation, intensity (HSI), hue, saturation, value (HSV), hue, saturation, lightness (HSL), any International Commission on Illumination (CIE) color space such as CIELAB, may be used. The one or more color image data frames and/or the skin region data sets may be converted from a first color space to a second color space, such as from RGB to CIELAB to enhance detectable color differences and improve accuracy of the liveness detection.

The extracting may be performed using (bit) mask(s) and/or array/matrix indexing. The extracting may be performed in-place, i.e., the locations of the skin region data sets are identified in the color image data frame(s), and subsequent processing of the skin region data sets is performed directly on the data of the color image data frame(s). Alternatively, or additionally, the skin region data sets may be excerpted from the color image data frame(s) e.g., by a copy operation, and subsequent processing of the skin region data sets is performed on the excerpted skin region data sets.

In an embodiment illustrated in FIG. 4, the skin regions are rectangular skin regions. At least one of the plurality of skin regions may be rectangular, or all of the plurality of skin regions may be rectangular. A benefit of the rectangular shape is more efficient processing of the skin region data sets, as they may be extracted using e.g., rectangular masks or array/matrix indexing. In an embodiment, the plurality of skin regions has, or each skin region has a predetermined size. This may further increase the efficiency of the processing and ease the comparing of color distributions in a further step of the method.

In addition to the rectangular shape, the skin regions may take any other shape. This allows for better representation of certain regions of the face, such as the under-eye regions, as they commonly extend over a crescent-shaped, non-rectangular area on the face. A skin region may have the same shape and/or size in a first frame and in a second frame, or the shape and/or size of the skin region may be different in a first frame than in a second frame. The size may be defined as a number of pixels and/or as a width and height in pixels, for example. This may allow better consideration for movement of the face and/or changes in zoom level between different frames.

The method of FIG. 3 further comprises computing 306 a plurality of color distributions, each color distribution being computed on the basis of one of the plurality of skin region data sets. Color distributions characterize the color content of a skin region with minimal loss of information, when compared to e.g., averaging of color values. A distribution type of the color distributions may be a probability density function, cumulative distribution function, probability distribution, histogram, local binary pattern histogram, or co-occurrence matrix, of pixels or data values of the respective skin region data set, for example.

Each color distribution of the plurality of color distributions may be of the same distribution type. The same type ensures that the plurality of color distributions may be reasonably compared with one another. Alternatively, the plurality of color distributions may comprise color distributions of different distribution types. For example, the plurality of color distributions may comprise at least two color distributions of a first distribution type, and at least two color distributions of a second distribution type. In this case, there are at least two color distributions of each type. This ensures that the color distributions of each (first and second) type may be compared with the other color distribution(s) of the same type. For example, the plurality of color distributions may comprise local binary pattern histograms (to capture texture information) and probability density functions.

The selection of the distribution type and the implementation of the computing may depend on the color space of the color image data frames and correspondingly the color space of the skin region data sets. As color image data usually contains multiple channels, such as the red, green and blue channels in RGB data, or the hue, saturation, value channels in HSV data, the color distributions may be multivariate distributions. For example, a multivariate probability density function may be computed for RGB data of a skin region data set. The (three) variables of the distribution are in this case the red, green and blue channels of the RGB data.

Referring again to FIG. 4, in an example wherein at least one skin region is identified in each of a plurality of color image data frames 402, 404, 406, a first right forehead skin region color distribution 422 may be computed based on a first right forehead skin region data set extracted from the right forehead skin region 412 of frame 402, a second right forehead skin region color distribution 426 may be computed based on a second right forehead skin region data set extracted from the right forehead skin region of frame 404, and a third right forehead skin region color distribution 430 may be computed based on a third right forehead skin region data set extracted from the right forehead skin region of frame 406.

In an example wherein a plurality of skin regions are identified in a single color image data frame 402, a right forehead skin region color distribution 422 may be computed based on a right forehead skin region data set extracted from the right forehead skin region 412 of frame 402, and a right cheek skin region color distribution 424 may be computed based on a right cheek skin region data set extracted from the right cheek skin region 416 of frame 402.

Continuing the previous example wherein now a plurality of skin regions are identified in a plurality of color image data frames 402, 404, a second right forehead skin region color distribution 426 may be computed based on a second right forehead skin region data set extracted from the right forehead skin region of frame 404, and a second right cheek skin region color distribution 428 may be computed based on a second right cheek skin region data set extracted from the right cheek skin region of frame 404. Further continuing the above example, a third right forehead skin region color distribution 430 may be computed based on a third right forehead skin region data set extracted from the right forehead skin region of frame 406, and a third right cheek skin region color distribution 432 may be computed based on a third right cheek skin region data set extracted from the right cheek skin region of frame 406.

The method of FIG. 3 further comprises determining 308 at least one distance between the plurality of color distributions. The distance may characterize the differences between the plurality of color distributions. When the at least one skin region is identified in each of a plurality of color image data frames 402, 404, 406 (as shown in FIG. 4), the at least one distance may be computed between color distributions in different frames. For example, a first distance between color distributions 422 and 426 in frames 402 and 404, respectively, may be computed. Further, a second distance between color distributions 426 and 430 in frames 402 and 404, respectively, may be computed. When the plurality of skin regions is identified in a single color image data frame, the at least one distance may be computed between color distributions of different skin regions. For example, a third distance between color distributions 422 and 424 in frame 402 may be computed.

When the plurality of skin regions is identified in a plurality of color image data frames 402, 404, the at least one distance may be computed between color distributions of different skin regions and/or different frames. This may include computing the first, second, and/or third distances as specified above.

Alternatively, or additionally, the at least one distance may be computed between a first color distribution computed on the basis of a first skin region in a first frame and a second color distribution computed on the basis of a second skin region in a second frame. Here, the first frame and the second frame are different frames, and the first skin region and the second skin region are different skin regions. As an example, a fourth distance between color distributions 422 and 428 may be computed.

The at least one distance may comprise a plurality of distances. The plurality of distances may of the same type, examples of which are given below. The plurality of distances may each be computed between a different pair of color distributions. For example, any combination of the above examples of the at least one distance may be computed. As another example, distances between some or all pairs of the color distributions 422, 424, 426, 428, 430, 432 of FIG. 4 may be computed.

When the plurality of color distributions comprises color distributions of different distribution types, each distance of the plurality of distances may be computed between color distributions of the same type. For example, when the plurality of color distributions comprises at least two color distributions of a first distribution type and at least two color distributions of a second distribution type, a first distance may be computed between the at least two color distributions of the first distribution type, and a second distance may be computed between the at least two color distributions of the second distribution type. The first distance and the second distance may of the same type or of different types of distances, examples of which are given below.

Various types of distances may be suitable for estimating the color difference between the skin regions. In an embodiment, the at least one distance is selected from the group comprising: Kullback-Leibler divergence, mean shift, Jeffreys divergence (also known as Jeffreys distance), Kolmogorov-Smirnov distance, and earth mover's distance. Preferably, the at least one distance may be Jeffreys divergence or Kolmogorov-Smirnov distance. One distance or a plurality of distances may be computed between a first color distribution and a second color distribution. For example, in the case of RGB data, a red distance may be computed between a first red color distribution representing the distribution of values in the red channel of a first skin region data set and a second red color distribution representing the distribution of values in the red channel of a second skin region data set, a green distance may be computed between a first green color distribution representing the distribution of values in the green channel of the first skin region data set and a second green color distribution representing the distribution of values in the green channel of the second skin region data set, and a blue distance may be computed between a first blue color distribution representing the distribution of values in the blue channel of the first skin region data set and a second blue color distribution representing the distribution of values in the blue channel of the second skin region data set.

If the at least one distance is greater than a liveness threshold, positive liveness of the subject is detected 310 as shown in the flowchart of FIG. 3. Else, negative liveness of the subject is detected 312. The liveness threshold may be a minimum distance between two color distributions. The liveness threshold may represent a minimum difference between color distributions that is required to ascertain that the subject is alive or that a (color difference representing a) trace of a pulse is detected in the subject's face. The liveness threshold may be determined using machine learning methods. A different liveness threshold may be determined for each type of distance, and/or for each different skin region, and/or for each different pair of skin regions.

When the at least one distance comprises a plurality of distances, the plurality of distances may be combined to an aggregate distance which is then compared to the liveness threshold. The combining may be performed e.g., by averaging or selecting a minimum/maximum value. Alternatively, each of the plurality of distances may be individually compared to the liveness threshold. The results of the comparison may then be combined e.g., such that if all or at least a predetermined number of the distances exceed the liveness threshold, the liveness threshold is considered exceeded. Alternatively, or additionally, a different liveness threshold may be determined for each skin region or pair of skin regions. The liveness threshold may comprise a skin region liveness threshold used for comparing color distributions of the same skin region in two sequential frames. For example, the liveness threshold may comprise an under-eye liveness threshold used for comparing color distributions of the under-eye skin region in two sequential frames. Alternatively, or additionally, the liveness threshold may comprise a first skin region-second skin region liveness threshold used for comparing respective color distributions of the first skin region and the second skin region in the same frame. For example, the liveness threshold may comprise a forehead-under-eye liveness threshold used for comparing respective color distributions of the forehead skin region and an under-eye skin region in the same frame.

The method of FIG. 3 further comprises outputting 314 the detected positive or negative liveness. The outputting may comprise writing the detected liveness to the at least one memory of the apparatus. Alternatively, or additionally, the outputting may comprise transmitting the detected liveness e.g., via the network 16 (see FIG. 1) and/or by the network interface 102 (see FIG. 2). For example, when the method is performed by the server 14 of FIG. 1, the server 14 may transmit the detected liveness to the user device 12 e.g., via the network 16 and/or by a network interface of the server 14. The user device 12 may receive the detected liveness via the network 16 and/or by a network interface of the user device 12. Alternatively, when the method id performed by the user device 12, the user device 12 may transmit the detected liveness to the server 14 e.g., via the network 16 and/or by a network interface of the user device. The server 14 may receive the detected liveness via the network 16 and/or by a network interface of the server 14.

In an embodiment, the apparatus 100 of FIG. 2 or the system of FIG. 1 comprises an interface configured to output the detected positive or negative liveness. The interface may be the above-mentioned network interface, and/or the interface may be a user interface 108 shown in FIG. 2. The user interface may comprise e.g., a display, a speaker, and/or a haptic output device configured to output the detected positive or negative liveness.

The output positive liveness may be received and used by a further computer program or module to authorize, authenticate, or grant the subject access to perform further steps. The output negative liveness may respectively be used to prevent access, authorization and/or authentication in view of a likely presentation attack.

In an embodiment illustrated in FIG. 4, the plurality of skin regions comprises a first skin region 412 and a second skin region 416, the first skin region being different from the second skin region. Different skin regions may visually depict the flow of oxygenated blood across the face differently. Lack of a color difference between the plurality of skin regions may be indicative of wearing a mask. Further, a reduced number of color image data frames may need to be processed to obtain a similar accuracy for the liveness detection compared to when only one skin region is examined. As a technical effect, the time taken for the liveness detection may be decreased. The performance of the above embodiment may be improved when used with near-infrared image data.

In an embodiment, the first skin region 412 is above an eye level of the subject, and the second skin region 416 is below the eye level of the subject. The eye level may be defined as a straight line passing through both eyes of the subject across a color image data frame. The first skin region, being above the eye level of the subject, may be e.g., a forehead skin region 410, 412. The second skin region, being below the eye level of the subject, may be e.g., an under-eye skin region 414, 416 or a cheek skin region 418, 420. There are fewer capillary veins on the forehead than directly under the eyes and on the cheeks. Further, the color changes caused by the pulse of the subject are visible on the forehead/above the eye level later than below the eye level. The potential for detecting a significant color change indicating liveness of the subject is therefore increased, improving the accuracy of the detection.

In an embodiment, the first skin region is above an eyebrow level of the subject. The eyebrow level may be defined as a straight line passing through both eyebrows of the subject across a color image data frame. The eyebrow level excludes the small area of skin between the eyes and the eyebrows. It may also ease identifying the first skin region as eyebrows are easily detectable as facial landmarks.

In an embodiment, the second skin region is above a mouth level of the subject. The mouth level may be defined as a straight line, possibly substantially parallel to the eye level and/or the eyebrow level, passing along the mouth of the subject across a color image data frame. In an embodiment, the second skin region is above a nose level of the subject. The nose level may be defined as a straight line, possibly substantially parallel to the mouth level, eye level and/or eyebrow level, passing through the nose of the subject across a color image data frame. Benefits of the above embodiments are exclusion of skin areas potentially covered by a beard or mustache, and wherein color changes are not as clearly visible as in other areas of the face.

In an embodiment, the method further comprises extracting a first skin region data set from the first skin region 412 of a single color image data frame 402; extracting a second skin region data set from the second skin region 416 of the single color image data frame 402; and computing a first skin region color distribution 422 on the basis of the first skin region data set; computing a second skin region color distribution 424 on the basis of the second skin region data set; and wherein determining the at least one distance comprises determining a distance between the first skin region color distribution 422 and the second skin region color distribution 424. The color difference between the two different skin regions in the same frame is thus determined. As discussed above, blood flow originating from a heart rate pulse reaches different parts of the face at different times. Therefore, the skin color change caused by the pulse occurs at different times in different areas. Two different areas are therefore in different phases with respect to one another, and the phase difference may be detected as a color difference.

In an embodiment, the plurality of skin regions comprises a third skin region 420, and the method further comprises: extracting a third skin region data set from the third skin region 420 of the single color image data frame 402; and computing a third skin region color distribution on the basis of the third skin region data set; wherein determining the at least one distance comprises determining at least one distance between the third skin region color distribution and at least one of the first skin region color distribution and the second skin region color distribution. The plurality of skin regions, or the first, second and third skin regions may comprise a forehead skin region, an under-eye skin region, and a cheek skin region. For example, the first skin region may be a forehead skin region 410, 412, the second skin region may be an under-eye skin region 414, 416, and the third skin region may be a cheek skin region 418, 420. Inclusion of a third different skin region may further improve the accuracy of the detection.

It is noted that further skin regions, such as a fourth, fifth, or sixth skin region, or in principle any number of skin regions, may be processed in the way described herein. Different skin regions, i.e., skin regions corresponding to different areas of the face, may be non-overlapping. The method is not limited to any number of skin regions, provided that the regions have sufficient area as indicated by the standard meaning of the term ‘region’.

In an embodiment, the one or more color image data frames comprise only one color image data frame 402. A technical effect is increased speed of liveness detection, as the time taken to perform the method may be reduced to the time for processing the single color image data frame.

In an embodiment, the one or more color image data frames comprise a plurality of color image data frames 402, 404, 406. The plurality of color image data frames may be sequential and/or consecutive. Color changes that occur over time may thus be captured in the color image data frames.

In an embodiment, the plurality of color image data frames comprise a first color image data frame 402 and a second color image data frame 404, and the method further comprises: extracting a first color image data set from a skin region 412 identified in the first color image data frame 402; extracting a second color image data set from the skin region 412 identified in the second color image data frame 404; computing a first color image color distribution 422 on the basis of the first color image data set; and computing a second color image color distribution 426 on the basis of the second color image data set; wherein determining the at least one distance comprises determining a distance between the first color image color distribution and the second color image color distribution. The color change that occurred in the skin region between the first frame and the second frame is captured by the distance between the first color image color distribution and the second color image color distribution and may be used to determine the liveness of the subject.

In an embodiment, a capture time of the second color image data frame 404 is within 0.3 to 0.5 seconds(s) of a capture time of the first color image data frame 402. The capture time refers to the time when a sensor (camera) has measured/captured the color image data frame. The capture time may be retrieved from metadata of a color image data frame. The second color image data frame may be captured after the first color image data frame, or the first color image data frame may be captured after the second color image data frame. The difference in the capture times represents approximately half of a pulse cycle of a typical heart rate of 60 to 100 beats per minute. Timing the first and second frames a half-cycle apart may bring out the greatest color difference in the skin region under analysis, increasing the accuracy of the liveness detection. Alternatively, or additionally, as pulse rates may vary and there may be other constraints to the timing in addition the pulse rate, the capture time of the second color image data frame may range from 0.1 s, 0.2 s, 0.3 s, or 0.4 s, to 0.5 s, 0.6 s, 0.7 s, 0.8 s, 0.9 s, or 1.0 s, of the capture time of the first color image data frame. The first and second frame may be but need not be consecutive frames.

As indicated in the above time ranges, the method may still perform more rapidly than liveness detection methods based on heart rate signal detection or challenge-response methods, which usually require several seconds worth of measurement data.

In an embodiment, the camera comprised in the apparatus or system is configured to capture the plurality of image data frames such that a capture interval between a first color image data frame and a second color image data frame is within one of the ranges described above. The first color image data frame and the second color image data frame may be consecutive frames. Alternatively, or additionally, the method may comprise selecting the plurality of color image data frames from frames captured by a camera such that a capture time of a selected color image data frame is within one of the above-mentioned ranges of a capture time of a consecutive selected color image data frame.

The above embodiments may evidently be applied to any number of frames, such that the interval between the capture times of two frames, from which color distributions are computed, and a distance between the color distributions is determined, and the distance is compared to the liveness threshold to detect the liveness, is within any of the above-mentioned ranges.

In an embodiment, the method further comprises extracting the one or more color image data frames from video data depicting the face of the subject. The video data may be raw or unencoded video data. Alternatively, when the video data is encoded video data, the method may comprise decoding the video data to obtain individual frames. The one or more color image data frames may be obtained from the frames of the unencoded/raw/decoded video data.

In an embodiment, the method further comprises acquiring a plurality of consecutive/sequential color image data frames 402, 404; and averaging the plurality of consecutive/sequential color image data frames to obtain the one or more color image data frames. The averaging is performed over time to acquire one color image data frame from a plurality of consecutive/sequential color image data frames. The averaging may be performed repeatedly, each time for a different plurality of consecutive/sequential color image data frames (the different pluralities may be overlapping), to obtain a plurality of color image data frames. The consecutive/sequential color image data frames may be images that have been taken sequentially, or consecutive or sequential frames of video data. In an embodiment, the averaging is performed only if a frame rate of the video data is greater than or equal to a frame rate threshold, and/or if an exposure time of the color image data frames is smaller than or equal to an exposure time threshold. The frame rate threshold may be 60 frames per second (fps). The exposure time threshold may be 1/60 s. A longer exposure allows for obtaining more color information from the skin region(s) of interest. The averaging may compensate for an exposure time that is not as long as would be desirable. The exposure time and/or the frame rate may be stored in metadata of a color image data frame and retrieved from there by the apparatus.

In the context of the averaging, the capture time of a color image data frame obtained by averaging a plurality of sequential/consecutive color image data frames may be an average of the capture times of the plurality of sequential/consecutive color image data frames. Alternatively, the capture time may be the earliest or the latest of the capture times of the plurality of sequential/consecutive color image data frames.

In an embodiment, the plurality of skin regions comprises a first skin region 412 and a second skin region 416, and the one or more color image data frames comprise a first color image data frame 402 and a second color image data frame 404, and the method further comprises: extracting a first color image first skin region data set from the first skin region 412 identified in the first color image data frame 402; extracting a first color image second skin region data set from the second skin region 416 identified in the first color image data frame 402; extracting a second color image first skin region data set from the first skin 412 region identified in the second color image data frame 404; computing a first color image first skin region color distribution 422 on the basis of the first color image first skin region data set; computing a first color image second skin region color distribution 424 on the basis of the first color image second skin region data set; and computing a second color image first skin region color distribution 426 on the basis of the second color image first skin region data set; wherein determining the at least one distance comprises determining a distance between the first color image first skin region color distribution 422 and the first color image second skin region color distribution 424, and determining a distance between the first color image first skin region color distribution 422 and the second color image first skin region color distribution 426.

In the above embodiment, a distance between color distributions of two different skin regions in one frame is determined, and a distance between color distributions of the same skin region in two different frames is determined. The two different skin regions in one frame and the same skin region in two different frames may or may not be overlapping, i.e. the skin region compared to another skin region in the same frame need not be the same skin region that is compared to another skin region in another frame. Both distances are subsequently compared to the liveness threshold as described earlier and may thus affect the detected liveness. This may improve the accuracy of the detection. As has been discussed in relation to other embodiments, the above embodiment may be further applied to a greater number of different skin regions, such as three, four, five, or more different skin regions in one frame, whose color distributions are determined and compared. Similarly, the embodiment may be further applied to a greater number of color image data frames, such as three, four, five, or more color image data frames, for which color distributions of the same skin region in the different frames are determined and compared.

In an embodiment, determining the at least one distance comprises determining a first skin region distance between a color distribution 422 of a first skin region 412 identified in a first color image data frame 402 and a color distribution 424 of a second skin region 416 identified in the first color image data frame 402; determining a second skin region distance between a color distribution 426 of the first skin region identified in a second color image data frame 404 and a color distribution 428 of the second skin region identified in the second color image data frame 404; and determining a frame distance between the first skin region distance and the second skin region distance. The frame distance may be a difference of the first skin region distance and the second skin region distance. The frame distance may be compared to the liveness threshold to detect the liveness of the subject.

In an embodiment, determining the at least one distance comprises determining a first frame distance between a color distribution 422 of a first skin region 412 identified in a first color image data frame 402 and a color distribution 426 of the first skin region identified in a second color image data frame 404; determining a second frame distance between a color distribution 424 of a second skin region 416 identified in a first color image data frame 402 and a color distribution 428 of the second skin region identified in the second color image data frame 404; and determining a skin region distance between the first frame distance and the second frame distance. The skin region distance may be a difference of the first frame distance and the second frame distance. The skin region distance may be compared to the liveness threshold to detect the liveness of the subject. The skin region distance may exceed the liveness threshold if there is a sufficient phase difference between the color changes in the first skin region and the second skin region, which is indicative of a pulse. This may allow for circumventing illumination changes or certain kinds of spoofing attempts.

In an embodiment, determining the at least one distance comprises determining a distance between a color distribution 422 of a first skin region 412 identified in a first color image data frame 402 and a color distribution 428 of a second skin region identified in a second color image data frame 404. That is, the color difference between two different skin regions in two different frames is determined. As has been discussed in relation to other embodiments, this and the above embodiments may be further applied to a greater number of different skin regions, such as three, four, five, or more different skin regions, and/or to a greater number of color image data frames, such as three, four, five, or more color image data frames.

If desired, the different functions discussed herein may be performed in a different order and/or concurrently with other. Furthermore, if desired, one or more of the above-described functions and embodiments may be optional or may be combined.

The embodiments and features, if any, described in this specification that do not fall under the scope of the independent claims are to be interpreted as examples useful for understanding various embodiments of the invention.

It is also noted herein that while the above describes example embodiments, these descriptions should not be viewed in a limiting sense. Rather, there are several variations and modifications, which may be made without departing from the scope of the present disclosure as defined in the appended claims.

1. A computer-implemented method for face liveness detection, the method comprising:
obtaining one or more color image data frames, each color image data frame depicting a face of a subject;
identifying a plurality of skin regions by one of:
a) identifying at least one skin region in each of a plurality of color image data frames;
b) identifying a plurality of skin regions in a single color image data frame;
c) identifying a plurality of skin regions in each of a plurality of color image data frames;
extracting a skin region data set from each one of the plurality of identified skin regions;
computing a plurality of color distributions, each color distribution being computed on the basis of one of the plurality of skin region data sets;
determining at least one distance between the plurality of color distributions;
if the at least one distance is greater than a liveness threshold, detecting positive liveness of the subject, and else detecting negative liveness of the subject; and
outputting the detected positive or negative liveness.

2. The method of claim 1, wherein the plurality of skin regions comprise a first skin region (412) and a second skin region, the first skin region being different from the second skin region.

3. The method of claim 2, wherein the first skin region is above an eye level of the subject, and the second skin region is below the eye level of the subject.

4. The method of claim 2, further comprising:
extracting a first skin region data set from the first skin region of a single color image data frame;
extracting a second skin region data set from the second skin region of the single color image data frame; and
computing a first skin region color distribution on the basis of the first skin region data set;
computing a second skin region color distribution on the basis of the second skin region data set;
wherein determining the at least one distance comprises determining a distance between the first skin region color distribution and the second skin region color distribution.

5. The method of claim 4, wherein the plurality of skin regions comprises a third skin region, and the method further comprises:
extracting a third skin region data set from the third skin region of the single color image data frame; and
computing a third skin region color distribution on the basis of the third skin region data set;
wherein determining the at least one distance comprises determining at least one distance between the third skin region color distribution and at least one of the first skin region color distribution and the second skin region color distribution.

6. The method of claim 1, wherein the one or more color image data frames comprise only one color image data frame.

7. The method of claim 1, wherein the one or more color image data frames comprise a plurality of color image data frames.

8. The method of claim 7, wherein the plurality of color image data frames comprise a first color image data frame and a second color image data frame, and wherein the method further comprises:
extracting a first color image data set from a skin region identified in the first color image data frame;
extracting a second color image data set from the skin region identified in the second color image data frame;
computing a first color image color distribution on the basis of the first color image data set; and
computing a second color image color distribution (426) on the basis of the second color image data set;
wherein determining the at least one distance comprises determining a distance between the first color image color distribution and the second color image color distribution.

9. The method of claim 8, wherein a capture time of the second color image data frame is within 0.3 to 0.5 seconds of a capture time of the first color image data frame.

10. The method of claim 1, further comprising:
extracting the one or more color image data frames from video data depicting the face of the subject.

11. The method of claim 1, further comprising:
acquiring a plurality of consecutive color image data frames; and
averaging the plurality of consecutive color image data frames to obtain the one or more color image data frames.

12. The method of claim 1, wherein the at least one distance is selected from the group comprising: Kullback-Leibler divergence, mean shift, Jeffreys divergence, Kolmogorov-Smirnov distance, and earth mover's distance.

13. The method of claim 1, wherein the plurality of skin regions comprises a first skin region and a second skin region, and wherein the one or more color image data frames comprise a first color image data frame and a second color image data frame, and wherein the method further comprises:
extracting a first color image first skin region data set from the first skin region identified in the first color image data frame;
extracting a first color image second skin region data set from the second skin region identified in the first color image data frame;
extracting a second color image first skin region data set from the first skin region identified in the second color image data frame;
computing a first color image first skin region color distribution on the basis of the first color image first skin region data set;
computing a first color image second skin region color distribution on the basis of the first color image second skin region data set; and
computing a second color image first skin region color distribution on the basis of the second color image first skin region data set;
wherein determining the at least one distance comprises determining a distance between the first color image first skin region color distribution and the first color image second skin region color distribution, and determining a distance between the first color image first skin region color distribution and the second color image first skin region color distribution.

14. An apparatus comprising at least one processor, at least one memory including computer program code, the at least one memory and the computer program code configured to, with the at least one processor, cause the apparatus to perform:
obtaining one or more color image data frames, each color image data frame depicting a face of a subject;
identifying a plurality of skin regions by one of:
a) identifying at least one skin region in each of a plurality of color image data frames;
b) identifying a plurality of skin regions in a single color image data frame;
c) identifying a plurality of skin regions in each of a plurality of color image data frames;
extracting a skin region data set from each one of the plurality of identified skin regions;
computing a plurality of color distributions, each color distribution being computed on the basis of one of the plurality of skin region data sets;
determining at least one distance between the plurality of color distributions;
if the at least one distance is greater than a liveness threshold, detecting positive liveness of the subject, and else detecting negative liveness of the subject; and
outputting the detected positive or negative liveness.

15. The apparatus of claim 14, further comprising a camera (107) configured to measure the one or more color image data frames, and an interface configured to output the detected positive or negative liveness.

16. A non-transitory computer-readable medium comprising computer program code configured to, when executed by at least one processor, cause an apparatus or a system to perform:
obtaining one or more color image data frames, each color image data frame depicting a face of a subject;
identifying a plurality of skin regions by one of:
a) identifying at least one skin region in each of a plurality of color image data frames;
b) identifying a plurality of skin regions in a single color image data frame;
c) identifying a plurality of skin regions in each of a plurality of color image data frames;
extracting a skin region data set from each one of the plurality of identified skin regions;
computing a plurality of color distributions, each color distribution being computed on the basis of one of the plurality of skin region data sets;
determining at least one distance between the plurality of color distributions;
if the at least one distance is greater than a liveness threshold, detecting positive liveness of the subject, and else detecting negative liveness of the subject; and
outputting the detected positive or negative liveness.
